(19) United States
(12) Plant Patent Application Publication
(10) Pub. No.: US 2014/0201876 P1
(43) Pub. Date: Jul. 17, 2014

(54) IMPATIENS PLANT NAMED 'DHAR329'

(50) ;

(71) Applicant: Gavriel Danziger, Moshav Mishmar Hashiva (IL)

(72) Inventor: Gavriel Danziger, Moshav Mishmar Hashiva (IL)

(21) Appl. No.: 13/694,923

(22) Filed: Jan. 17, 2013

(51) Int. Cl. A01H 5/00

(52) U.S. Cl. PLT/318.6

(57) ABSTRACT

A new and distinct Impatiens cultivar named ‘DHAR329’ is disclosed, characterized by large violet flowers. The new variety has good branching, and is sun resistant. The new variety is a Impatiens, normally produced as an outdoor garden or container plant.

### LATIN NAME OF THE GENUS AND SPECIES

Impatiens hawkeri

### VARIETY DENOMINATION

‘DHAR329’

### BACKGROUND OF THE INVENTION

The new Impatiens cultivar is a product of a planned breeding program conducted by the inventor, Gavriel Danziger, in Moshav Mishmar Hashiva, Israel. The objective of the breeding program was to produce new Impatiens varieties for ornamental commercial applications. The cross resulting in this new variety was made during July of 2009.

The seed parent is the unpatented, proprietary seedling variety referred to as Impatiens hawkeri ‘2807’ The pollen parent is the unpatented, proprietary seedling variety referred to as Impatiens hawkeri ‘1803’. The new variety was discovered in July of 2010 by the inventor in a group of seedlings resulting from the 2009 crossing, in a research greenhouse in Moshav Mishmar Hashiva, Israel.

Asexual reproduction of the new cultivar was performed by leaf cuttings. The cuttings are from an apical bud that has no more than two expanded leaves and three to four immature leaves present. This was first performed at a research greenhouse in Moshav Mishmar Hashiva, Israel in July of 2010 and has shown that the unique features of this cultivar are stable and reproduced true to type in successive generations.

### SUMMARY OF THE INVENTION

The cultivar ‘DHAR329’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DHAR329’ These characteristics in combination distinguish ‘DHAR329’ as a new and distinct Impatiens cultivar:
1. Large flower size.
2. Violet flower color.
3. Good branching.
4. A sun resistant plant.

Plants of the new cultivar ‘DHAR329’ are similar to plants of the seed parent, Impatiens hawkeri ‘NG-Z-116’ in most horticultural characteristics, however, plants of the new cultivar ‘DHAR329’ are more vigorous in growth habit and have darker foliage than the seed parent. ‘DHAR329’ also produces a violet flower whereas the seed parent produces a white flower.

Plants of the new cultivar ‘DHAR329’ are similar to plants of the pollen parent; Impatiens hawkeri ‘NG-8-7741’ in most horticultural characteristics, however, plants of the new cultivar ‘DHAR329’ produce smaller flowers and are more vigorous growth in habit than the pollen parent.

### Commercial Comparison

Plants of the new cultivar ‘DHAR329’ are comparable to the variety Impatiens ‘Sunpatiens Vigorous Magenta’ U.S. Plant Pat. No. 17,662. The two Impatiens varieties are similar in most horticultural characteristics; however, the new variety ‘DHAR329’ differs in having larger, darker colored flowers and a more compact growth habit than the comparator.

Plants of the new cultivar ‘DHAR329’ can also be compared to the commercial variety Impatiens ‘Sunpatiens Compact Magenta’ U.S. Plant Pat. No. 21,769. These varieties are similar in most horticultural characteristics; however ‘DHAR329’ produces a larger flower and is more vigorous in its growth habit.

### BRIEF DESCRIPTION OF THE PHOTOGRAPHS

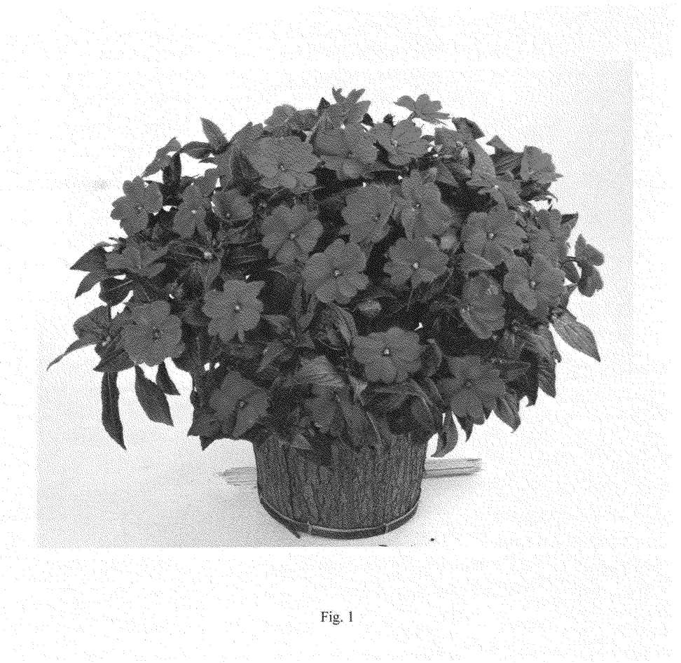

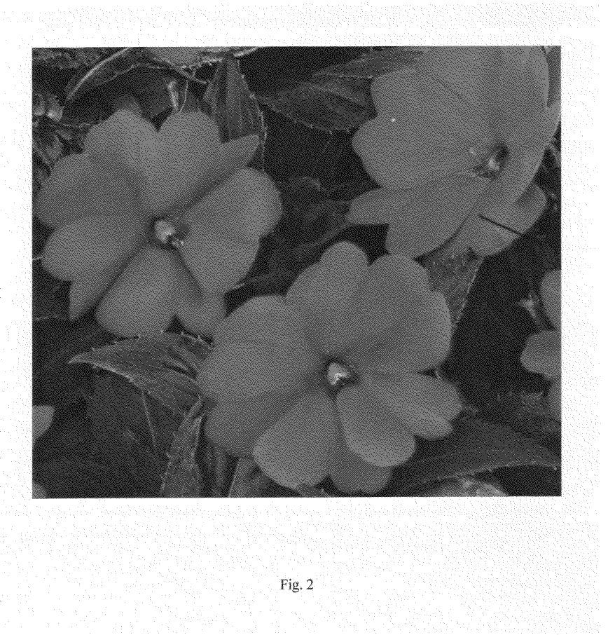

The accompanying photograph in FIG. 1 illustrates in full color a typical plant of ‘DHAR329’ grown in a greenhouse, in a 16 cm pot. Age of the plant photographed is approximately 14 weeks from a rooted cutting. FIG. 2 illustrates in full color a close up of a typical bloom of ‘DHAR329’. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001 except where general terms of ordinary dictionary significance are used. The following observations and measurements describe ‘DHAR329’ plants grown outdoors during, Spring, Summer, and Autumn in Moshav Mishmar Hashiva, Israel. The growing temperature ranged from 20° C. to 35° C. during the day and from 15° C. to 25° C. during the night. General light conditions are bright, normal sunlight. Measurements and numerical values represent averages of typical plant types.
Botanical classification: Impatiens hawkeri ‘DHAR329’
Propagation:
Time to initiate roots: Summer: About 10 to 12 days at approximately 25° C. Winter: About 14 to 20 days at approximately 18° C.
Root description: Fine, adventitious fibrous.
Plant:
Age of plant described: Approximately 14 weeks from rooted cutting.
Pot size of plant described: 16 cm.
Growth habit: Mounded
Height: 25 cm.
Plant spread: 40 cm.
Growth rate: Vigorous
Branching characteristics: Highly branched.
Length of primary lateral branches: 23 cm.
Quantity of primary lateral branches: 5-7
Characteristics of primary lateral branches:
   Form.—Oval shape.
   Diameter.—1 cm.
   Color.—Yellow Green group 146 C RHS.
   Texture.—Smooth.
   Strength.—Strong, very stable.
Internode length: 6 cm.
Foliage:
Leaf:
   Arrangement.—Alternate.
   Quantity.—Approximately 20-25 per branch.
   Average length.—6.5 cm.
   Average width.—3 cm.
   Shape of blade.—Lanceolate to ovate.
   Apex.—Attenuate.
   Base.—Acuminate.
   Margin.—Ciliate.
   Texture of top surface.—Smooth.
   Pubescence.—None.
   Aspect.—45 degrees.
   Color.—Green Young foliage upper side: Green Group 143 A RHS Young foliage under side: Yellow Green Group 146 DRHS Mature foliage upper side: Yellow Green Group 147 A RHS Mature foliage under side: Yellow Green Group 147 C RHS.
   Venation.—Midrib with secondary veins. Pinnate, only lower surface Type: Pinnate. Venation color upper side: Yellow Green Group 147 C RHS Venation color under side: Red Group 39 B RHS.
   Petiole.—Length: 1 cm. Diameter: 0.5 cm. Color: Yellow Green Group 145 C RHS Texture: Smooth.
Flower:
Natural flowering season: Flowers at any day length. All year round in a greenhouse environment. Flowering is continuous from Spring until Fall in an outdoor garden.
Days to flowering from rooted cutting: 10 weeks.
Inflorescence and flower type and habit: Single flowers raised above the foliage. The corolla consists of five petals.
Rate of flower opening: 10 to 15 days from bud to fully opened flower.
Flower longevity on plant: 5-8 Days
Persistent or self-cleaning: Self cleaning
Bud:
   Shape.—Ovate.
   Length.—2 cm.
   Diameter.—1 cm.
   Color.—Purple Group N78C RHS.
Flower size:
   Diameter.—7.5-8 cm.
   Depth.—Approximately 1.5 cm.
Petals:
   Quantity.—5.
   Arrangement.—overlapping.
   Length.—3.5-4 cm.
   Width.—4 cm.
   Shape.—Obcordate.
   Apex.—Obcordate.
   Base.—Broad attenuate.
   Margin.—Entire.
   Texture, upper and lower surfaces.—Smooth.
   Color.—purple When opening, upper surface: Red Purple Group N74A RHS When opening, lower surface: Red Purple Group N74B RHS Fully opened, upper surface: Red Purple Group N74A RHS Fully opened, lower surface: Purple Pink Group N74C RHS.
Spurs:
   Quantity per flower.—1.
   Length.—5 cm.
   Diameter.—0.1 cm.
   Aspect.—Spur curvature 40°-45° from peduncle.
   Color.—Towards base: Red Purple Group 59D RHS Apex: Red Purple Group 59B RHS.
Sepals:
   Quantity.—2.
   Length.—2 cm.
   Width.—1 cm.
   Shape (overall).—Oblong Apex: Acute Base: Rounded.
   Margin.—Entire.
   Color.—Yellow Green Group 145B RHS.
Peduncles:
   Length.—4.5-5 cm.
   Diameter.—0.5 cm.
   Angle.—About 45° to the lateral branch.
   Strength.—Stable, flexible.
   Texture.—Smooth.
   Color.—Yellow Green Group 145 D RHS.
Fragrance: None
Reproductive organs:
Stamens: There are five stamens with introrse anthers that are more or less fused and form a cap over the ovary.
   Number.—5.
Anthers:
   Shape.—Oblong.
   Length.—Approximately 0.4 cm.
   Color.—Red Purple Group 72 C RHS.
   Pollen.—Color: White Group 155 D RHS Quantity: Few.
Pistil:
   Number.—1.
   Length.—0.5 cm.
   Style.—Length: 0.5 cm. Color: Yellow Green Group 146 C RHS.
   Stigma.—Shape: Round Color: White Group N 155B RHS Ovary Color: Yellow-Green group, RHS 144D RHS.
Other characteristics:
Seeds and fruits: 5-10 seeds per fruit
Disease/pest resistance: Common to cultivar
Temperature tolerance: Tolerant at 30° C. and tolerant at 10° C. degrees Celsius (grows best at 10° C.-30° C.)

What is claimed is:

1. A new and distinct cultivar of Impatiens plant named ‘DHAR329’ as herein illustrated and described.
